(19) United States
(12) Patent Application Publication
(10) Pub. No.: US 2009/0295274 A1
(43) Pub. Date: Dec. 3, 2009

(54) Deuterated Semiconducting Organic Compounds for Use in Light-Emitting Devices

(76) Inventors: Kuo-Chu Hwang, Hsinchu (TW); Chun-Chih Tong, Hsinchu (TW); Tzu-Hao Yeh, Hsinchu (TW)

Correspondence Address: WPAT, PC;INTELLECTUAL PROPERTY ATTORNEYS, 7225 BEVERLY ST., ANNANDALE, VA, 22003, US

(21) Appl. No.: 12/025,091

(22) Filed: Feb. 4, 2008

(51) Int. Cl. H01J 1/63; C07D 403/10; C07D 491/16; C07D 471/06; C07F 1/00; C07F 3/00; C07F 5/00; C07F 7/00

(52) U.S. Cl. 313/504; 548/440; 546/66; 546/94; 546/7

(57) ABSTRACT

The present invention discloses deuterated semiconducting organic compounds. The deuterated semiconducting organic compounds comprise at least one partially or fully deuterated non-conjugated portion linked to the conjugated portion. The mentioned deuterated semiconducting organic compounds can be used in optoelectronic devices, such as light-emitting devices and photodiodes, with enhanced performance and lifetime. The deuterated semiconducting organic compounds of this application can be employed as emissive layer, charge-transporting layer, or energy transfer material in organic light-emitting devices.

### BACKGROUND OF THE INVENTION

1. Field of the Invention

The present invention is generally related to semiconducting organic compounds for use in optoelectronic devices, and more particularly to deuterated semiconducting organic compounds for use in light-emitting devices.

2. Description of the Prior Art

Organic light emitting diodes (OLEDs) are under intensive investigation because of their potential of achieving improved device performances. OLED has the advantages of self-light, wide view angles, being lightweight, fast response time, and low power consumption, etc. Although having many advantages, organic light-emitting materials still suffer from some drawbacks, such as low light emission efficiency and poor high-voltage stability. In view of the above matter, developing a novel organic compound having high voltage stability and low turn-on voltage to prolong the usage lifetime of the device and to increase luminance efficiency is still an important task for the industry.

### SUMMARY OF THE INVENTION

According to the above, the present invention provides new deuterated semiconducting organic compounds for using in light-emitting devices to fulfill the requirements of this industry.

One object of the present invention is to employ deuterated semiconducting organic compounds for using in light-emitting devices. By partially or fully deuterated the protons of the semiconducting organic compounds, the high voltage stability of the light-emitting devices can be increased, and the turn-on voltage can be decreased. Thus, the usage lifetime of the light-emitting devices can be prolonged efficiently.

Another object of the present invention is to employ deuterated semiconducting organic compounds for using in light-emitting devices. By partially or fully deuterated the protons of the semiconducting organic compounds, the external quantum efficiency and the light emission efficiency of the light-emitting materials can be increased, and thus, the luminance efficiency of the light-emitting devices can be improved.

According to above-mentioned objectives, the present invention discloses deuterated semiconducting organic compounds for using in light-emitting devices. The deuterated semiconducting organic compounds comprise at least one conjugated portion, and at least one non-conjugated portion linked to the conjugated portion. The protons linked to the non-conjugated portion are partially or fully deuterated. The light-emitting device comprises a pair of electrodes and one or more organic layers disposed between the electrodes. The mentioned deuterated semiconducting organic compounds are employed in the organic layers. Mostly, the deuterated semiconducting organic compounds are used as host materials or guest materials in the light-emitting device.

### BRIEF DESCRIPTION OF THE DRAWINGS

FIG. 1a˜1c show external quantum efficiency as a function of current density in a device of ITO/(H- or D-)Q₂AlOAr (50 nm)/Alq₃ (30 nm)/Mg:Ag (10:1), wherein the voltage was applied from 0 to 18 V and back to 0 V for consecutively two cycles for each device, and the EL maximum is located at 490 nm for the protonated device and at 500 nm for the deuterated device as designated in the figures.

### DESCRIPTION OF THE PREFERRED EMBODIMENTS

What is probed into the invention is organometallic complexes and their application. Detail descriptions of the structure and elements will be provided in the following in order to make the invention thoroughly understood. Obviously, the application of the invention is not confined to specific details familiar to those who are skilled in the art. On the other hand, the common structures and elements that are known to everyone are not described in details to avoid unnecessary limits of the invention. Some preferred embodiments of the present invention will now be described in greater detail in the following. However, it should be recognized that the present invention can be practiced in a wide range of other embodiments besides those explicitly described, that is, this invention can also be applied extensively to other embodiments, and the scope of the present invention is expressly not limited except as specified in the accompanying claims.

One preferred embodiment of this present invention discloses small molecular deuterated semiconducting organic compounds, wherein the deuterated semiconducting organic compounds is not polymers. The deuterated semiconducting organic compounds comprise at least one conjugated portion, and at least one non-conjugated portion linked to the conjugated portion. The protons of the non-conjugated portion are fully or partially deuterated. In some examples, the protons of the conjugated portion are also deuterated.

The non-conjugated portion of the deuterated semiconducting organic compounds is selected from the group consisted of C₁˜C₃₀ linear alkyl, C₁˜C₃₀ branch alkyl, C₁˜C₃₀ cyclic alkyl, C₁˜C₃₀ alkoxyl, C₁˜C₃₀ silyl. In one preferred example of this embodiment, the semiconducting organic compound further comprises a metal, wherein said metal is selected from Li, Na, K, Be, Mg, Ca, Ti, Cr, Mo, Mn, Fe, Ru, Os, Co, Rh, Ir, Ni, Pd, Pt, Cu, Zn, Cd, B, Al, Ga, In, Si, N and P.

The deuterated semiconducting organic compounds can be used in optoelectronic devices, such as light-emitting devices, and photodiodes, with enhanced performance and lifetime. In one example of this embodiment, the deuterated semiconducting organic compounds can be employed as emissive layer, charge-transporting layer, or energy transfer material in organic light-emitting devices, wherein the conjugated portion of the deuterated semiconducting organic compounds is used as chromophore.

Another preferred embodiment of the present invention discloses deuterated semiconducting organic compounds for using in optoelectronic devices. The general structure of the deuterated semiconducting organic compounds comprises at least one conjugated portion and at least one non-conjugated portion. The protons of the non-conjugated portion are partially or fully deuterated. The non-conjugated portion of the deuterated semiconducting organic compounds is selected from C₁˜C₃₀ linear alkyl, C₁˜C₃₀ branch alkyl, C₁˜C₃₀ cyclic alkyl, C₁˜C₃₀ alkoxyl, C₁˜C₃₀ silyl.

In one preferred example of this embodiment, the protons of the conjugated portion are also partially or fully deuterated. In another preferred example of this embodiment, the deuterated semiconducting organic compounds comprise at least one metal. The metal is selected from Li, Na, K, Be, Mg, Ca, Ti, Cr, Mo, Mn, Fe, Ru, Os, Co, Rh, Ir, Ni, Pd, Pt, Cu, Zn, Cd, B, Al, Ga, In, Si, N and P.

The mentioned deuterated semiconducting organic compounds can be applied in organic electroluminescence devices, organic phosphorescence devices, solar cells or other organic optoelectronic devices.

In one preferred example of this embodiment, the deuterated semiconducting organic compounds are used in light-emitting devices. The above light-emitting device comprises a pair of electrodes, and one or more organic layers disposed between the electrodes. The organic layers comprise a light-emitting layer, and at least one of the organic layers comprises the deuterated semiconducting organic compounds. The deuterated semiconducting organic compounds comprise at least one conjugated portion as the chromophore of the light-emitting device, and at least one non-conjugated portion linked to the conjugated portion. The protons of the non-conjugated portion are partially or fully deuterated. In some examples, the protons of the conjugated portion are partially or fully deuterated. Furthermore, the deuterated semiconducting organic compounds can further comprise at least one metal selected from Li, Na, K, Be, Mg, Ca, Ti, Cr, Mo, Mn, Fe, Ru, Os, Co, Rh, Ir, Ni, Pd, Pt, Cu, Zn, Cd, B, Al, Ga, In, Si, N and P. According to this example, the deuterated semiconducting organic compounds can be employed as the host materials, or the guest materials in the light-emitting device. In one example of this embodiment, the mentioned deuterated semiconducting organic compounds can be used in light-emitting device such as organic light-emitting diode (OLED) or polymer light-emitting diode (PLED).

The preferred examples of the structure and fabricating method for the deuterated semiconducting organic compounds according to the application are described in the following. However, the scope of this application should be based on the claims, but is not restricted by the following examples.

### General Deuteration Procedure:

The deuteration of 8-hydroxyquinoline, 2-methyl-8-hydroxyquinoline, and 2,6-dimethylphenol was achieved by the following procedures. [Keyes, T. E.; O'Connor, C. M.; O'Dwyer, U.; Coates, C. G.; Callaghan, P.; McGarvey, J. J.; Vos, J. G. J. Phys. Chem. A 1999, 103, 8915; and Keyes, T. E.; Weldon, F.; Muller, E.; Pechy, P.; Gratzel, M.; Vos, J. G. J. Chem. Soc., Dalton Trans. 1995, 16, 2075]Briefly, 500 mg of 8-hydroxyquinoline (or other compounds) was dissolved/dispersed in a mixed solution containing 30 mL of D₂O, 5 mL of acetone-d6, and 0.5 g of a catalyst Pd/C (10% Pd, Aldrich) in a Teflon-coated stainless steel high-pressure reactor, which was then heated in an oven at 220° C. for 48-72 h. After reaction, the stainless steel reactor was allowed to cool to room temperature. The solid catalyst was filtered off and washed by dichloromethane and acetone for a few times. The product was collected by vacuum removal of the solvents. The percentage of deuteration was determined by FTIR spectroscopy, mass spectrometry, and 1H NMR spectroscopy.

In some examples according to this application, the above products are further used to synthesize metal complex by the following procedures. [Tang, C. W.; VanSlyke, S. A. Appl. Phys. Lett. 1987, 51, 913; and Curioni, A.; Boero, M.; Andreoni, W. Chem. Phys. Lett. 1998, 294, 263] The deuterated compounds were purified by a train-sublimation process in an oven under a temperature gradient and a pressure of 10⁻³ Torr and recrystallized in ethanol. Compounds were identified by NMR spectroscopy, mass spectrometry, and X-ray crystallography.

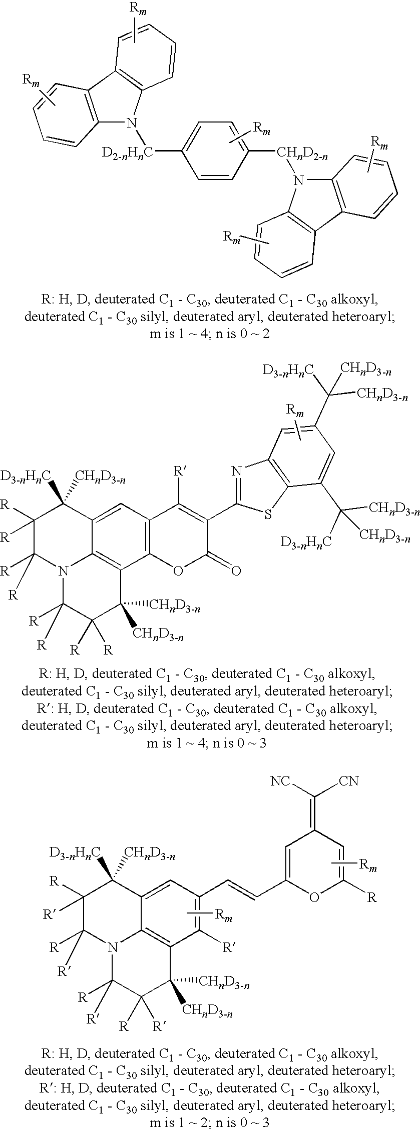

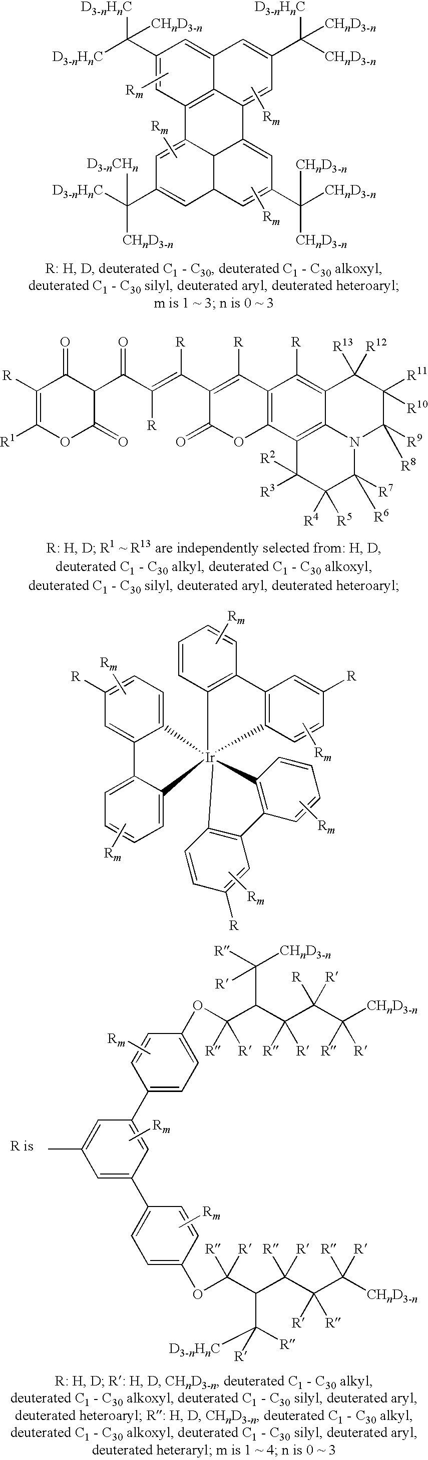

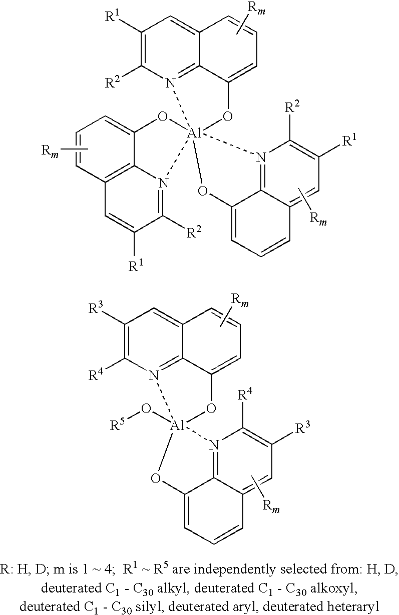

### Procedures for Measurements of Fluorescence Lifetime and Quantum Yields:

Luminescence quantum yields (Φ_{F}) of H-Alq₃ and D-Alq₃ were determined using fluorescein (Φ_{F}) 0.90, in 0.1 M NaOH) as a standard under a degas condition. Fluorescein has two absorption maxima at 320 and 496 nm. The absorbances of fluorescein and Alq₃ in N,N-dimethylformamide (DMF) at 320 nm were adjusted to be the same when measuring the fluorescence quantum yield. The quantum yields, measured under a degas condition in DMF, were 0.11 and 0.19 for H-Alq₃ and D-Alq₃, respectively. H-Alq₃ and D-Alq₃ show the same UV-visible absorption and photoluminescence (PL) spectra. Fluorescence lifetime measurements were carried out in a single-photon counter (Edinburgh, model OB900, England). The fluorescence quantum yields of H- and D-Q₂AlOAr were measured in a similar way.

### OLED Device Fabrication:

The ITO glass (Merck) with 80 nm thickness of ITO was ultrasonically cleaned in an aqueous solution, followed by a patterning process. Electroluminescence materials were then deposited onto the patterned ITO glass in a thermal evaporator at a pressure of 5×10⁻⁶ Torr. The cathode consisted of Mg:Ag (10:1, total 55 nm) by coevaporation of Mg and Ag metals at a deposition rate of 5-7 and 0.5-0.7 Å s⁻¹, respectively. The current-voltage-luminescence (I-V-L) measurements were carried simultaneously using a Keithly 2400 Source meter and a Newport 1835-C optical meter with a Newport 818-ST silicon photodiode as the detector. The electroluminescence (EL) spectra of as-fabricated devices were measured on a Hitachi F-4500 luminescence spectrometer. The deuterated and protonated devices for the light emitter were fabricated in the same batch at the same day to avoid any contribution from variation in the fabrication process. The fabrication of devices was repeated at least three times to warrantee the reproducibility of the reported phenomena.

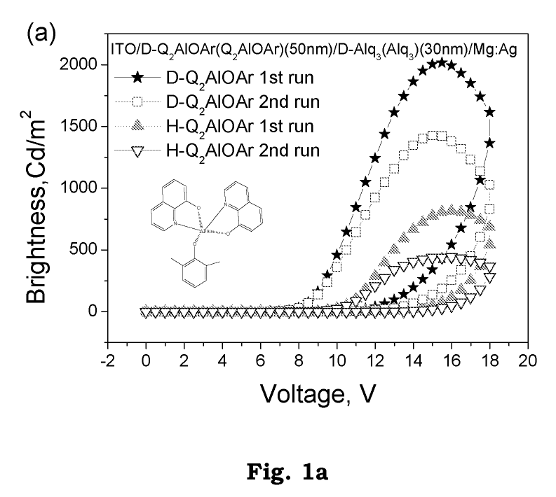

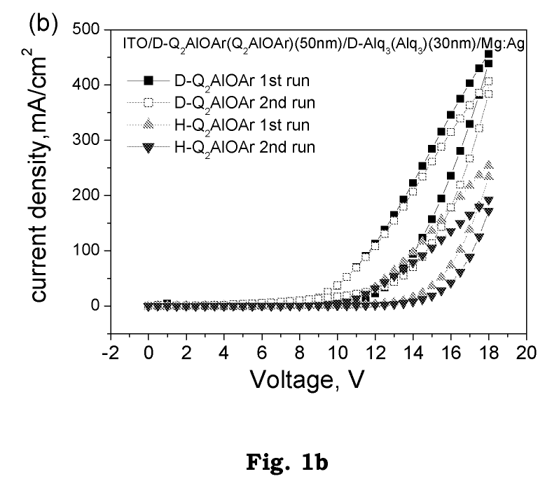

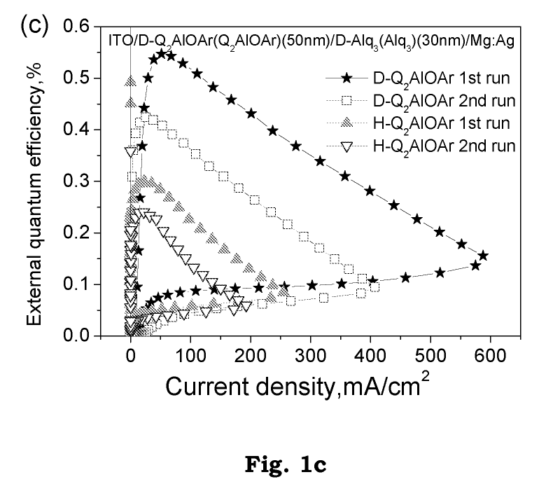

In one preferred example of this application, H-Q₂AlOAr and D-Q₂AlOAr are used as the semiconducting organic compounds and following the mentioned process to fabricate the light emitting devices. The Electroluminescence properties of the devices are shown as FIG. 1a˜1c.

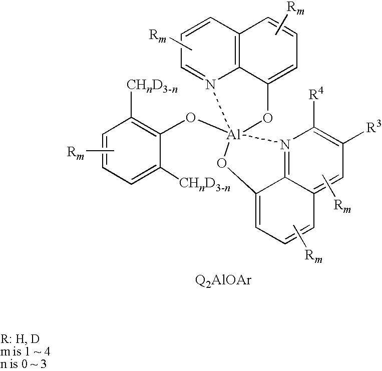

As shown in FIG. 1a and FIG. 1b, the EL intensities and current densities increase almost linearly at higher applied voltages. The EL intensities, however, drop quickly upon the applied voltage passing a critical value of ˜15.2 V for D-Q₂AlOAr and 15.8 V for H-Q₂AlOAr, respectively. The external quantum efficiency of the D-Q₂AlOAr device is higher than that of the H-Q₂AlOAr device at all current densities, 1.9-fold at 50 mA/cm² and ˜2.8-fold at 150 mA/cm². After experiencing the high-voltage-induced degradation in the first cycle, the external quantum efficiency of the D-Q₂AlOAr device becomes 3.5-fold of the H-Q₂AlOAr device at 150 mA/cm². And the D-Q₂AlOAr device loses 35% external quantum efficiency in the second forward (low-to-high) half cycle at 150 mA/cm², which is smaller than the 49% loss in the H-Q₂AlOAr device. The slightly lower critical high voltage of 15.2 V for D-Q₂AlOAr is due to a higher current density of the deuterated device itself. In the above green and blue EL devices, it was also noticed that the deuterated devices have lower turn-on voltages in both green (by ˜0.8 V) and blue (by ˜2.5 V) devices. The cause for lower turn-on voltages in deuterated devices could be a result of higher light-emitting efficiencies of the deuterated light-emitting materials.

To sum up, the present application discloses deuterated semiconducting organic compounds for using in optoelectronic devices, especially in light-emitting devices. The light-emitting device comprises a pair of electrodes, and one or more organic layers disposed between the electrodes. At least one of the organic layers comprises the deuterated semiconducting organic compounds. By partially or fully modifying the protons of the semiconducting organic compounds with deuterium, the high voltage stability of the light-emitting devices can be increased, and the turn-on voltage can be decreased. Therefore, the usage lifetime of the light-emitting devices can be prolonged efficiently. Furthermore, by employing the deuterated semiconducting organic compounds If this application, the external quantum efficiency and the light emission efficiency of the light-emitting materials can be increased, and the luminance efficiency of the light-emitting devices can be efficiently improved.

Obviously many modifications and variations are possible in light of the above teachings. It is therefore to be understood that within the scope of the appended claims the present invention can be practiced otherwise than as specifically described herein. Although specific embodiments have been illustrated and described herein, it is obvious to those skilled in the art that many modifications of the present invention may be made without departing from what is intended to be limited solely by the appended claims.

What is claimed is:

1. A light-emitting device comprising:
a pair of electrodes and one or more organic layers disposed between said electrodes, said one or more organic layers comprising a light-emitting layer, wherein at least one of said one or more organic layer comprises a semiconducting organic compound with a conjugated portion and at least one non-conjugated portion linked to the conjugated portion, wherein protons of said non-conjugated portion are partially or fully deuterated.

2. The light-emitting device according to claim 1, wherein said non-conjugated portion is selected from the group consisted of C₁˜C₃₀ linear alkyl, C₁˜C₃₀ branch alkyl, C₁˜C₃₀ cyclic alkyl, C₁˜C₃₀ alkoxyl, C₁˜C₃₀ silyl.

3. The light-emitting device according to claim 1, wherein the protons of the conjugated portion are partially or fully deuterated.

4. The light-emitting device according to claim 1, wherein the semiconducting organic compound further comprises a metal, wherein said metal is selected from Li, Na, K, Be, Mg, Ca, Ti, Cr, Mo, Mn, Fe, Ru, Os, Co, Rh, Ir, Ni, Pd, Pt, Cu, Zn, Cd, B, Al, Ga, In, Si, N and P.

5. The light-emitting device according to claim 1, wherein the semiconducting organic compound is used as the host material in the light-emitting device.

6. A small molecular deuterated semiconducting organic compound for using in an optoelectronic device, comprising:
at least one conjugated portion, wherein the protons of the conjugated portion are partially or fully deuterated; and
at least one non-conjugated portion linked to said conjugated portion, wherein the protons of the non-conjugated portion are partially or fully deuterated.

7. The small molecular deuterated semiconducting organic compound according to claim 6, wherein said non-conjugated portion is selected from the group consisted of C₁˜C₃₀ linear alkyl, C₁˜C₃₀ branch alkyl, C₁˜C₃₀ cyclic alkyl, C₁˜C₃₀ alkoxyl, C₁˜C₃₀ silyl.

8. The small molecular deuterated semiconducting organic compound according to claim 6, further comprises a metal, wherein said metal is selected from Li, Na, K, Be, Mg, Ca, Ti, Cr, Mo, Mn, Fe, Ru, Os, Co, Rh, Ir, Ni, Pd, Pt, Cu, Zn, Cd, B, Al, Ga, In, Si, N and P.

9. The small molecular deuterated semiconducting organic compound according to claim 6, wherein the conjugated portion is used as chromophore in a light emitting device.
